(19) United States
(12) Patent Application Publication
(10) Pub. No.: US 2010/0256194 A1
(43) Pub. Date: Oct. 7, 2010

(54) PHARMACEUTICAL FORMULATIONS COMPRISING NITROCATECHOL DERIVATIVES AND METHODS OF MAKING THE SAME

(75) Inventors: Teófilo Cardoso de Vasconcelos, Sao Mamede do Coronado (PT); Ricardo Jorge dos Santos Lima, Sao Mamede do Coronado (PT); Pedro Miguel da Costa Barrocas, Sao Mamede do Coronado (PT); Ligia Sofia de Castro Pereira, Sao Mamede do Coronado (PT); Rui Cerdeira de Campos Costa, Sao Mamede do Coronado (PT)

Correspondence Address: O'BRIEN JONES, PLLC, 1951 Kidwell Drive, Suite 550 B, Tysons Corner, VA, 22182, US

(73) Assignee: BIAL - Portela & Ca, S.A., Sao Mamede do Coronado (PT)

(21) Appl. No.: 12/750,957

(22) Filed: Mar. 31, 2010

Related U.S. Application Data
(60) Provisional application No. 61/165,794, filed on Apr. 1, 2009.

(51) Int. Cl. A61K 31/4439; A61P 25/16; A61P 25/14; A61P 9/12

(52) U.S. Cl. 514/340

(57) ABSTRACT

The present disclosure relates to compositions and pharmaceutical formulations comprising at least one active pharmaceutical ingredient chosen from nitrocatechol derivatives of formula I as defined herein and salts, esters, hydrates, solvates and other derivatives thereof and methods of making the same.

### FIELD OF THE DISCLOSURE

The present disclosure relates to compositions and pharmaceutical formulations comprising at least one active pharmaceutical ingredient chosen from nitrocatechol derivatives and salts thereof.

### BACKGROUND

Levodopa (L-DOPA) has been used in clinical practice for several decades in the symptomatic treatment of various conditions, including Parkinson's disease. L-DOPA is able to cross the blood-brain barrier, where it is then converted to dopamine and increases the levels thereof. However, conversion of L-DOPA to dopamine may also occur in the peripheral tissue, possibly causing adverse effects upon administration of L-DOPA. Therefore, it has become standard clinical practice to co-administer a peripheral amino acid decarboxylase (AADC) inhibitor, such as carbidopa or benserazide, which prevents conversion to dopamine in peripheral tissue.

This has led to an interest in the development of inhibitors of the enzyme catechol-O-methyltransferase (COMT) based on the hypothesis that inhibition of the enzyme may provide clinical improvements in patients afflicted with Parkinson's disease undergoing treatment with L-DOPA, since COMT catalyses the degradation of L-DOPA.

It has been found, as set forth in International Publication Nos. WO 2007/013830 and WO 2007/117165, which are incorporated herein by reference, that compounds of formula I disclosed herein, which are nitrocatechol derivatives, are potent and long-acting COMT inhibitors. Those compounds are both bioactive and bioavailable. Thus, compounds of formula I have potentially valuable pharmaceutical properties in the treatment of some central and peripheral nervous system disorders where inhibition of O-methylation of catecholamines may be of therapeutical benefit, such as, for example, mood disorders, Parkinson's disease and disorders, restless leg syndrome, gastrointestinal disturbances, edema formation states, and hypertension. Furthermore, these compounds may also have activity in treating other diseases and disorders, not related to the inhibition of O-methylation of catecholamines.

It has also been found, however, that the compounds of formula I may be sensitive to certain excipients, which may, cause decomposition of the compounds of formula I and/or lack of stability of the compositions and formulations containing these compounds. The compounds of formula I may also exhibit a low bulk density and/or poor flow characteristics, which may increase the difficulty in formulating and/or manufacturing a stable dosage formulation containing the active compound.

The inventors have now discovered compositions and formulations thereof comprising at least one active pharmaceutical ingredient (“API”) chosen from nitrocatechol derivatives of formula I as defined herein and salts, esters, hydrates, solvates and other derivatives thereof. In at least one embodiment, the API is present in granular form. In further embodiments, the compositions and/or formulations may also comprise at least one filler and at least one binder. In various embodiments when the API is present in granular form, the at least one filler and at least one binder may, independently, be intragranular (i.e., granulated with the API and/or contained within the same granules as the API), extragranular (i.e., present outside the granules of API), or part intragranular and part extragranular. In yet further embodiments of the present disclosure, the compositions may exhibit a bulk density that is greater than that of the API alone, and that may, in certain embodiments, be a significantly increased. The compositions may also exhibit improvements in other characteristics such as compressibility. Use of the methods described herein may also result in improvements in the granule properties of the compositions such as improved granule size and uniformity of granule size and/or of granule mass. In yet further embodiments, the compositions and/or formulations may be stable over time and under different conditions, and may, in certain embodiments exhibit enhanced stability.

### SUMMARY

In accordance with the detailed description and various exemplary embodiments described herein, the present disclosure relates to compositions and formulations thereof comprising at least one API chosen from nitrocatechol derivatives of formula I as defined herein and salts, esters, hydrates, solvates and other derivatives thereof. In various exemplary embodiments, the at least one nitrocatechol derivative is 2,5-dichloro-3-(5-(3,4-dihydroxy-5-nitrophenyl)-1,2,4-oxadiazol-3-yl)-4,6-dimethylpyridine 1-oxide or 5-[3-(2,5-dichloro-4,6-dimethylpyridin-3-yl)-[1,2,4]oxadiazol-5-yl]-3-nitrobenzene-1,2-diol. The at least one nitrocatechol derivative may also be a mixture of 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol.

In at least one embodiment, the API may be present in granular form. In some embodiments, the compositions and/or formulations may comprise a further API, for example the compositions and/or formulations may comprise, in addition to the at least one API chosen from nitrocatechol derivatives of formula I, further APIs such as L-DOPA, a peripheral amino acid decarboxylase (AADC) inhibitor, such as carbidopa or benserazide. In further embodiments, the compositions and/or formulations may also comprise at least one filler and at least one binder. In additional embodiments, the filler may not be a phosphate derivative and/or the binder may not be a polyvinylpyrrolidone (“PVP”) derivative compound. In various embodiments when the API is granular, the at least one filler and at least one binder may, independently, be intragranular, extragranular, or part intragranular and part extragranular. In yet further embodiments of the present disclosure, the compositions may exhibit a bulk density that is greater than that of the API alone, and that may, in certain embodiments, be a significantly increased. The compositions may also exhibit improvements in other characteristics such as compressibility. Use of the methods described herein may also result in improvements in the granule properties of the compositions such as improved granule size and uniformity of granule size and/or of granule mass. In yet further embodiments, the compositions and/or formulations may be stable over time, and may, in certain embodiments exhibit enhanced stability.

### DETAILED DESCRIPTION

It is to be understood that both the foregoing general description and the following detailed description are exemplary and explanatory only, and are not restrictive of the invention as claimed. Other embodiments of the invention will be apparent to those skilled in the art from consideration of the specification and practice of the invention disclosed herein.

In various embodiments, the present disclosure relates to compositions and formulations thereof comprising at least one API chosen from nitrocatechol derivatives of formula I as defined herein and salts, esters, hydrates, solvates and other derivatives thereof, at least one filler, and at least one binder. In a further embodiment, the at least one filler is not a phosphate derivative and/or the at least one binder is not a PVP derivative compound. In at least one further embodiment, the API may be present in granular form.

As used herein, the term “granules,” “granular form,” “API granules” and variations thereof, are intended to include the particles produced by wet or dry granulation of the API chosen from nitrocatechol derivatives of formula I as defined herein and salts, esters, hydrates, solvates and other derivatives thereof. In various embodiments of the present disclosure, the API may comprise two or more nitrocatechol derivatives of formula I, for example the composition may comprise 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol. In additional embodiments, the granules may further comprise at least one filler and/or at least one binder.

As used herein, the term “composition,” and variations thereof, is intended to mean a composite comprising at least one API chosen from nitrocatechol derivatives of formula I as defined herein and salts, esters, hydrates, solvates and other derivatives thereof, at least one filler, and at least one binder. In certain embodiments, the composition may comprise two or more nitrocatechol derivatives of formula I (i.e. APIs), for example the composition may comprise 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol, at least one phosphate derivative, and at least one PVP derivative compound. In various embodiments, the composition may comprise granules of the at least one API, and the at least one filler and at least one binder may be independently intragranular (i.e., granulated with the API and/or contained within the same granules as the API), extragranular (i.e., present outside the granules of API, or part intragranular and part extragranular. For example, the filler may be 10 wt % to 90 wt %, 20 wt % to 80 wt %, 30 wt % to 70 wt %, 40 wt % to 60 wt %, or about 50 wt % intragranular, with the remaining portion being extragranular. The binder may be 10 wt % to 90 wt %, 20 wt % to 80 wt %, 30 wt % to 70 wt %, 40 wt % to 60 wt %, or about 50 wt % intragranular, with the remaining portion being extragranular. In a further exemplary embodiment, the composition may further comprise at least one excipient, and in yet further embodiments, the composition may be appropriate for filling a capsule, making a tablet and/or for directly administering to patients, for example packaged in sachets.

As used herein, the terms “formulation,” “pharmaceutical formulation,” and variations thereof, are intended to include compositions described herein that are further processed or formulated into a dosage form. By way of example only, in various exemplary embodiments, the formulations may comprise a composition described herein, typically in the form of granules, in a dosage form suitable for administration to a subject, such as a capsule or compressed dosage form such as a tablet. In a further exemplary embodiment, the formulations may comprise a composition described herein, typically in the form of granules, mixed with at least one excipient in a dosage form suitable for administration to a subject, such as a capsule or compressed dosage form such as a tablet.

As used herein, the nitrocatechol derivatives of formula I are defined as follows:

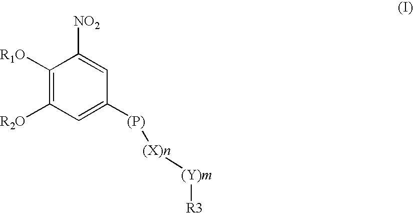

wherein:
R₁ and R₂ are independently selected from hydrogen or a group which is hydrolysable under physiological conditions, optionally substituted lower alkanoyl or aroyl;
X is a methylene group;
Y is an atom of oxygen, nitrogen, or sulphur,
n is selected from 0, 1, 2, and 3;
m is 0 or 1;
R₃ is a pyridine group chosen from the formulas A, B, C, D, E and F which is connected as indicated by the unmarked bond:

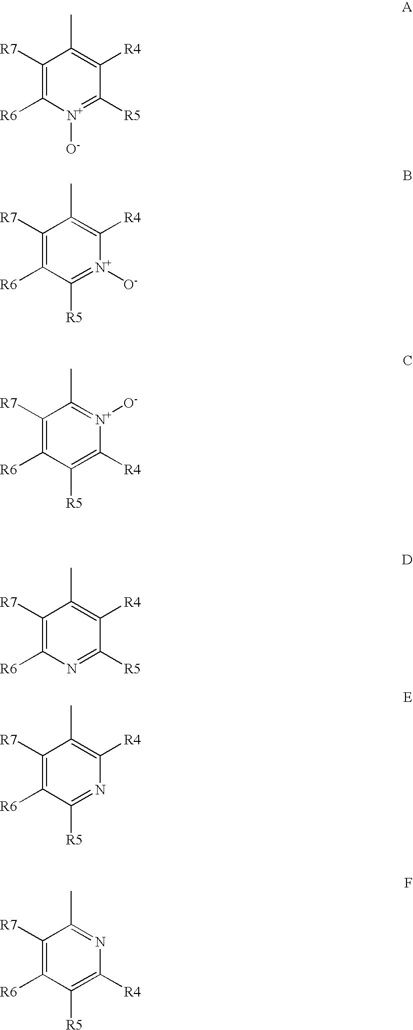

wherein:

R₄, R₅, R₆, and R₇ are independently chosen from hydrogen, C₁-C₆-alkyl, C₁-C₆-thioalkyl, C₁-C₆-alkoxy, C₆-C₁₂-aryloxy or a C₆-C₁₂-thioaryl group, C₁-C₆-alkanoyl or C₇-C₁₃-aroyl group, amino, C₁-C₆-alkylamino, C₁-C₆-dialkylamino, C₃-C₁₂-cycloalkylamino, C₃-C₁₂-heterocycloalkylamino, C₁-C₆-alkylsulphonyl, C₆-C₁₂-arylsulphonyl, halogen, C₁-C₆-haloalkyl, e.g., trifluoromethyl, cyano, nitro or a heteroaryl group; or two or more of residues R₄, R₅, R₆ and R₇ taken together represent aliphatic or heteroaliphatic rings or aromatic or heteroaromatic rings; and

P is a central unit, for example a planar unit, such as those selected from the regioisomers of 1,3,4-oxadiazol-2,5-diyl; 1,2,4-oxadiazol-3,5-diyl; 4-methyl-4H-1,2,4-triazol-3,5-diyl; 1,3,5-triazin-2,4-diyl; 1,2,4-triazin-3,5-diyl; 2H-tetrazol-2,5-diyl; 1,2,3-thiadiazol-4,5-diyl; 1-alkyl-3-(alkoxycarbonyl)-1H-pyrrol-2,5-diyl wherein alkyl is represented by methyl, ethyl, n-propyl and n-butyl and wherein alkoxy is represented by methoxy, ethoxy, n-propoxy and isopropoxy; 1-alkyl-1H-pyrrol-2,5-diyl wherein alkyl is represented by methyl, ethyl, n-propyl and n-butyl; thiazol-2,4-diyl; 1-H-pyrazol-1,5-diyl; pyrimidin-2,4-diyl; oxazol-2,4-diyl; carbonyl; 1H-imidazol-1,5-diyl; isoxazol-3,5-diyl; furan-2,4-diyl; 3-alkoxycarbonylfuran-2,4-diyl wherein alkoxy is represented by methoxy, ethoxy, n-propoxy, and isopropoxy; benzene-1,3-diyl; and (Z)-1-cyanoethen-1,2-diyl. Suitable groups which are hydrolysable under physiological conditions are well known in the art and include groups that form, with the O atom, an ether, ester, carbonic acid, or an ester linkage.

In one exemplary embodiment, P is chosen from 1,3,4-oxadiazol-2,5-diyl and 1,2,4-oxadiazol-3,5-diyl.

In a further exemplary embodiment, the at least one nitrocatechol derivative of formula I is 2,5-dichloro-3-(5-(3,4-dihydroxy-5-nitrophenyl)-1,2,4-oxadiazol-3-yl)-4,6-dimethylpyridine 1-oxide or 5-[3-(2,5-dichloro-4,6-dimethylpyridin-3-yl)-[1,2,4]oxadiazol-5-yl]-3-nitrobenzene-1,2-diol.

As described herein, the compositions and/or formulations of the disclosure comprise at least one API chosen from nitrocatechol derivatives of formula I and salts, esters, hydrates, solvates and other derivatives thereof. In various embodiments, the at least one API is chosen from nitrocatechol derivatives of formula I and salts, esters, hydrates, solvates and other derivatives thereof wherein P is chosen from 1,3,4-oxadiazol-2,5-diyl and 1,2,4-oxadiazol-3,5-diyl. In various further exemplary embodiments, the at least one API is 2,5-dichloro-3-(5-(3,4-dihydroxy-5-nitrophenyl)-1,2,4-oxadiazol-3-yl)-4,6-dimethylpyridine 1-oxide. In other exemplary embodiments, the at least one API is 5-[3-(2,5-dichloro-4,6-dimethylpyridin-3-yl)-[1,2,4]oxadiazol-5-yl]-3-nitrobenzene-1,2-diol.

The at least one nitrocatechol derivative of formula I may also be a mixture of 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol.

In embodiments where the at least one nitrocatechol derivative is a mixture of two nitrocatechol derivatives, such as 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol, the ratio of the two components may be approximately 50:50 or any variation thereof, such as approximately 60:40, 70:30, 80:20, 90:10, 95:5, 97:3, or 99:1, or the proportion of one of the nitrocatechol derivatives may be present in an amount up to and including 5%, up to an including 3% or up to and including 1% of the amount of the other nitrocatechol, for example 5-[3-(2,5-dichloro-4,6-dimethylpyridin-3-yl)-[1,2,4]oxadiazol-5-yl]-3-nitrobenzene-1,2-diol may be present in an amount of up to and including 5%, up to and including 3% or up to and including 1% of the amount of 2,5-dichloro-3-(5-(3,4-dihydroxy-5-nitrophenyl)-1,2,4-oxadiazol-3-yl)-4,6-dimethylpyridine 1-oxide.

The at least one API chosen from nitrocatechol derivatives of formula I as disclosed herein, and salts, esters, hydrates, solvates and other derivatives thereof, may exhibit low bulk density, thereby making it difficult to formulate and manufacture a dosage form. For example, 2,5-dichloro-3-(5-(3,4-dihydroxy-5-nitrophenyl)-1,2,4-oxadiazol-3-yl)-4,6-dimethylpyridine 1-oxide, a nitrocatechol of formula I, may exhibit a bulk density of less than 0.1 g/ml prior to granulation and/or formulation, and 5-[3-(2,5-dichloro-4,6-dimethylpyridin-3-yl)-[1,2,4]oxadiazol-5-yl]-3-nitrobenzene-1,2-diol may exhibit a bulk density of around 0.2 g/ml prior to granulation and/or formulation, as determined by the method described hereinbelow.

Formulating APIs of low bulk density gives rise to many problems. For example poor content uniformity, particle segregation, little or no flowability, high average weight variability, capping and lamination of tablets and high friability of tablets.

In at least one exemplary embodiment, the amount (or dosage) of the at least one API present in the compositions and/or formulations may be a therapeutically effective amount. As used herein, “therapeutically effective amount” means an amount of a therapeutic agent sufficient to treat, alleviate, and/or prevent any condition treatable and/or preventable by administration of a composition of the disclosure, in any degree. That amount can, for example, be an amount sufficient to exhibit a detectable therapeutic or preventative or ameliorative effect. The effect may include, for example, treatment, alleviation, and/or prevention of the conditions listed herein. The actual amount required, e.g. for treatment of any particular patient, will depend upon a variety of factors including the disorder being treated and/or prevented; its severity; the specific pharmaceutical composition employed; the age, body weight, general health, gender, and diet of the patient; the mode of administration; the time of administration; the route of administration; the rate of excretion of the therapeutic agent; the duration of the treatment; any drugs used in combination or coincidental with the therapeutic agent; and other such factors well known to those skilled in the art. In various embodiments, for example, a formulation, i.e, a capsule or tablet dosage form, may contain 1 mg or more of API, for example 2.5 mg or more, 5 mg or more, 10 mg or more, 20 mg or more, 40 mg or more, 50 mg or more, or 100 mg or more of API. The API content in the formulation can vary from 0.02 wt % to 90 wt %, for example from 0.1 wt % to 70 wt %, from 0.2 wt % to 50 wt %, or from 0.3 wt % to 45 wt %.

The at least one filler of the present disclosure includes, but is not limited to, calcium carbonate, cellulose powder, silicified microcrystalline cellulose, cellulose acetate, compressible sugar, confectioner's sugar, dextrane, dextrin, dextrose, fructose, kaolin, lactitol, lactose, magnesium carbonate, magnesium oxide, maltodextrin, maltose, mannitol, microcrystalline cellulose, polydextrose, simethicone, sodium alginate, sodium chloride, sorbitol, starches, pregelatinized starch, sucrose, trehalose, and xylitol.

In various embodiments of the present disclosure, the at least one filler is not a phosphate derivative. As used herein, the term “phosphate derivative,” and variations thereof, is intended to mean substances comprising calcium phosphate, including, but not limited to: calcium phosphate, dibasic anhydrous (for example, A-TAB™, Di-Cafos A-N™, Emcompress™ Anhydrous, and Fujicalin™); calcium phosphate, dibasic dihydrate (for example, Cafos™, Calipharm™, Calstar™, Di-Cafos™, Emcompress™); and calcium phosphate tribasic (for example, Tri-Cafos™, TRI-CAL™ WG, TRI-TAB™). In a further embodiment, the at least one filler may be chosen from starches, lactose, and cellulose. In at least one embodiment, at least two fillers may be present, for example a combination of starch, lactose, and/or cellulose.

In various embodiments, for example, the at least one filler may constitute 0.5 wt % to 99.5 wt % of the composition and/or formulation, for example, 20 wt % to 95 wt %, 40 wt % to 95 wt %, 40 wt % to 85 wt %, 40 wt % to 70 wt %, 60 wt % to 95 wt %, or 80 wt % to 95 wt % of the total weight of the composition and/or formulation. The filler may be intragranular, extragranular or part intragranular and part extragranular. By way of example, a composition and/or formulation may comprise 85 wt % filler. The amount of the at least one filler will vary depending, in part, upon the desired dosage, bulk density, and stability of the composition and/or formulation.

The at least one binder of the present disclosure includes, but is not limited to, acacia, alginic acid, carbomer, carboxymethylcellulose sodium, ceratonia, cottonseed oil, dextrin, dextrose, gelatin, guar gum, hydrogenated vegetable oil type I, hydroxyethyl cellulose, hydroxyethylmethyl cellulose, hydroxypropyl cellulose, low substituted hydroxypropyl cellulose, hypromellose, magnesium aluminium silicate, maltodextrin, maltose, methylcellulose, ethylcellulose, microcrystalline cellulose, polydextrose, polyethylene oxide, polymethacrylates, sodium alginate, starch, pregelatined starch, stearic acid, sucrose and zein.

In various embodiments of the present disclosure, the at least one binder is not a PVP derivative compound. As used herein, the term “PVP derivative compound” and variations thereof, is intended to mean substances comprising polyvinyl pyrrolidone (PVP) and substituted versions thereof, including, but not limited to: povidone (for example, plasdone and kollidon); copovidone (for example, plasdone S-630™ and kollidon VA-64™); and cross-linked PVP (for example crospovidone). In a further embodiment, the at least one binder may be chosen from starches, and in at least one embodiment, it may be starch 1500™.

In various embodiments, the at least one binder may constitute 0.5 wt % to 40 wt % of the composition and/or formulation, for example, 1 wt % to 25 wt %, 5 wt % to 20 wt %, 8 wt % to 15 wt %, or 10 wt % to 15 wt % of the total weight of the composition and/or formulation. The binder may be intragranular, extragranular or part intragranular and part extragranular. By way of example only, a composition and/or formulation may comprise between 6 wt % and 8 wt % binder, such as 7 wt % or 6.3 wt % binder. The amount of the at least one binder will vary depending, in part, upon the desired dosage, bulk density, and stability of the resulting composition and/or formulation.

In one exemplary embodiment, the composition and/or formulation comprises 0.2 to 50 wt % API, 5 to 10 wt % binder, and 33 to 85 wt % filler, such as the following compositions and/or formulations:

| | | |
|---|---|---|
| API | 0.2-50 | wt % |
| Filler | 35.0-85.0 | wt % |
| Binder | 1.0-15.0 | wt % |
| Lubricants | 1.0-15.0 | wt % |
| Disintegrant | 1.0-15.0 | wt % |
| API | 30.0-50.0 | wt % |
| Filler | 35.0-60.0 | wt % |
| Binder | 3.0-10.0 | wt % |
| Lubricants | 1.0-10.0 | wt % |
| Disintegrant | 3.0-10.0 | wt % |
| API | 0.2-35 | wt % |
| Filler | 50.0-85.0 | wt % |
| Binder | 3.0-10.0 | wt % |
| Lubricants | 1.0-10.0 | wt % |
| Disintegrant | 3.0-10.0 | wt % |

The invention also includes a method of making a composition or formulation of the invention comprising the steps of:
granulating at least one active pharmaceutical ingredient chosen from 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol and salts thereof to form granules;
mixing at least one filler with the at least one active pharmaceutical ingredient before, during or after granulation;
mixing at least one binder with the at least one active pharmaceutical ingredient before, during or after granulation; and
preparing a pharmaceutical formulation in the form of a dosage form.

In various embodiments, the filler is not a phosphate derivative and/or the binder is not a polyvinylpyrrolidone (“PVP”) derivative compound.

In various exemplary embodiments of the present disclosure, the at least one API, at least one filler, and at least one binder are combined by mixing (also referred to herein as blending). The appropriate apparatus and mixing time and rate may easily be determined by those of skill in the art based on, for example, the amount of material present, the type of mixing process used, and other parameters known to those of skill in the art. For example, in various embodiments, the components may be mixed manually, using a V-blender, a high shear mixer, or any other mixing apparatus and/or process known to those of skill in the art. As a further example, in various embodiments, the components may be mixed for any appropriate period of time, such as 1 to 20 minutes or 2 to 10 minutes.

In various exemplary embodiments, the mixture may be dry or wet granulated. In at least one embodiment, the granules are wet-granulated using at least one granulation liquid. By way of example, the at least one granulation liquid may be chosen from water, ethanol, isopropanol, and/or acetone. In at least one embodiment, the granulation liquid may be water. The appropriate apparatus and mixing time and rate for granulation may be determined by those of skill in the art based on, for example, the amount of material and the amount of granulation liquid, if present. For example, in various embodiments, the components may be granulated manually, using a high shear mixer, planetary mixer or any other granulator apparatus and/or process known to those of skill in the art. As a further example, in various embodiments, the components may be granulated for any appropriate period of time, such as 1 to 60 minutes or 2 to 30 minutes. Determination of the endpoint of granulation is within the capability of the skilled person but can be determined by observance of stabilization of granule size and particle cohesion resulting in a decrease in air trapped inside the granule, or by attainment of steady state of rheological or correlated determination of voltage, conductivity torque, power consumption or near IR techniques. As a further example, granulation speeds may vary from 5% to 100% of the granulator mixing speed, such as from 25% to 100%.

In at least one exemplary embodiment, after the wet-granulation process is complete, the granules may then be dried. Granules may be dried to loss on drying (LOD) values below 6.0%, such as below 5% or between 1-3%. A suitable method for calculating LOD is described hereinbelow. The appropriate drying apparatus and drying time and temperature may be determined by those of skill in the art based on, for example, the amount of material present, moisture content of the material, and the granulation liquid. As non-limiting examples, a fluid bed dryer or tray dryer may be used, for example at a temperature of 25° C. or higher, 40° C. or higher, or 70° C. or higher, to dry the granules. For example, the granules may be dried at a temperature of 66° C.

In various exemplary embodiments, the granules may be sieved. Sieving the granules may separate out granules of a particular particle size, and may be used to select particles of an advantageous size for formulating a dosage form or manufacturing a dosage form. In various embodiments, the granules may be sieved over a screen or sieve of 0.5 mm or larger, for example a 0.6 mm, 0.8 mm, 1.0 mm and 1.6 mm screen.

In various exemplary embodiments, the composition may further include at least one additional excipient which may be blended with the at least one API, at least one filler and at least one binder before, during or after granulation. For example, in at least one embodiment, the at least one additional excipient may be chosen from, but is not limited to, excipients such as disintegrants, glidants, and lubricants.

Disintegrants of the present disclosure include, but are not limited to, agar, calcium carbonate, alginic acid, calcium phosphate (tribasic), carboxymethylcellulose calcium, carboxymethylcellulose sodium, colloidal silicon dioxide, croscarmellose sodium, crospovidone, docusate sodium, guar gum, low substituted hydroxypropyl cellulose, magnesium aluminum silicate, methylcellulose, microcrystalline cellulose, sodium alginate, sodium starch glycolate, polacrilin potassium, silicified microcrystalline cellulose, starch and pre-gelatinized starch, and mixtures thereof. In at least one embodiment, the disintegrant is a combination of disintegrants and/or at least two disintegrants are present, for example a combination of sodium starch glycolate and sodium carboxymethyl starch, such as that sold under the trade name Explotab™.

In various embodiments, the disintegrant may constitute 0.5 wt % to 40 wt % of the composition and/or formulation, for example, 1 wt % to 25 wt %, 5 wt % to 20 wt %, 10 wt % to 15 wt %, or 5 wt % to 15 wt %. By way of example, a composition and/or formulation may comprise between 6 wt % and 9 wt % disintegrant, such as 6.8 wt % disintegrant. The amount of the at least one disintegrant will vary depending, in part, upon the desired dosage, bulk density, and stability of the resulting composition and/or formulation.

Glidants of the present disclosure include, but are not limited to: calcium silicate, cellulose, powdered, colloidal silicon dioxide, magnesium silicate, magnesium trisilicate, starch, and talc, and mixtures thereof.

In various embodiments, the glidant may constitute 0.1 wt % to 15 wt % of the composition and/or formulation, for example, 0.5 wt % to 15 wt %, 1 wt % to 10 wt %, or 2 wt % to 6 wt %. The amount of glidant will vary depending, in part, upon the desired dosage, bulk density, and stability of the resulting composition and/or formulation.

Lubricants of the present disclosure include, but are not limited to: calcium stearate, glycerine monostearate, glyceryl behenate, glyceryl palmitostearate, hydrogenated castor oil, hydrogenated vegetable oil type I, magnesium lauryl sulphate, magnesium stearate, medium-chain triglycerides, poloxamer, polyethylene glycol, sodium benzoate, sodium chloride, sodium lauryl sulphate, sodium stearyl fumarate, stearic acid, talc, sucrose stearate, and zinc stearate, and mixtures thereof.

In various embodiments, lubricants may constitute 0.1 wt % to 15 wt % of the composition and/or formulation, for example, 0.5 wt % to 15 wt %, 1 wt % to 10 wt %, 1 wt % to 2 wt %, or 2 wt % to 8 wt %. The amount of lubricant will vary depending, in part, upon the desired dosage, bulk density, and stability of the resulting composition and/or formulation.

In various exemplary embodiments, the at least one excipient may be added before, during or after mixing of the at least one API and before (prior to) or during granulation and, thus may be an intragranular excipient. In other exemplary embodiments, the at least one excipient may be added to the composition after granulation, for example by blending with the granules, and thus may be present as an extragranular excipient. In various exemplary embodiments, at least one first excipient may be added prior to or during granulation and at least one second excipient and/or more of the at least one first excipient may be added to the composition after granulation. For example, in various embodiments, disintegrants may be added prior to or during granulation, whereas lubricants and glidants may be added after granulation.

In various exemplary embodiments, the composition comprising at least one API, at least one filler, and at least one binder may be used to make a formulation, such as, for example, to fill capsules or to form tablets.

Capsules for use in the present disclosure include, but are not limited to, gelatin capsules and hydroxypropylmethyl cellulose (hypromellose) capsules. Suitable methods for filling such capsules with a composition according to an embodiment of the disclosure are well-known to those of skill in the art.

Tablets of the present disclosure may be formed by any method known to those of skill in the art such as direct compression. In at least one embodiment of the present disclosure, the tablets may be coated, for example with aqueous based film-coatings, solvent based film-coatings and/or sugar coatings.

The formulations of the invention may also be colored, for example by inclusion of a coloring in the composition of the invention and/or by coating the composition and/or formulation.

In at least one embodiment of the present disclosure, the formulation is a capsule comprising at least one API, at least one filler, and at least one binder, optionally in granular form, and may further comprise at least one glidant and/or at least one disintegrant. In at least one embodiment of the present disclosure, the formulation is a tablet comprising at least one API, at least one filler, and at least one binder, optionally in granular form, and may further comprise at least one glidant, at least one lubricant, and/or at least one disintegrant.

In various exemplary embodiments of the present disclosure, the compositions may exhibit improved bulk density and/or flow properties relative to those of the API alone. As used herein, the terms “improved bulk density,” “significantly improved bulk density,” and variations thereof mean that the bulk density of the composition is approximately at least double, least three times, at least four times, or at least five times that of the API alone. It is within the ability of one of skill in the art to determine the bulk density of a compound or composition using methods generally accepted in the art. However, suitable methods include, for example, the European Pharmacopeia edition 6, Test 2.9.15 “apparent volume,” pages 285-286, EDQM, 2007, and USP 31, vol. 1, test <616> page 231-232, The United States Pharmacopeia Convention, 2008. Another example of a suitable method is described below:

Apparatus:
settling apparatus capable of producing in 1 minute 250±15 taps from a height of 3±0.2 mm. The support for the graduated cylinder with its holder, has a mass of 450±5 g
a 250 ml graduated cylinder (2 ml intervals) with a mass of 220±40 g

Method: Into a dry cylinder, introduce without compacting, 100.0 g (m g) of the test substance. Secure the cylinder in its holder. Read the unsettled apparent volume (V₀) to the nearest milliliter. Carry out 10, 500 and 1250 taps and read the corresponding volumes V₁₀, V₅₀₀, V₁₂₅₀, to the nearest milliliter. If the difference between V₅₀₀ and V₁₂₅₀ is greater than 2 ml, carry out another 1250 taps.

Alternatively, if it is not possible to select 100.0 g, select a test sample of any mass but with a volume between 50 ml and 250 ml, measure its apparent volume, V₀ as described above, and weigh the sample and specify the mass in the expression of results. Bulk/apparent density may then be determined in g/ml using the following formula:

m/V₀

where m is the mass in grams and V₀ the unsettled apparent volume.

Tapped apparent density may then be determined in g/ml using the following formula:

M/V₁₂₅₀

where m is the mass in grams and V₁₂₅₀ the apparent volume after 1250 hubs.

For example, as set forth above, 2,5-dichloro-3-(5-(3,4-dihydroxy-5-nitrophenyl)-1,2,4-oxadiazol-3-yl)-4,6-dimethylpyridine 1-oxide, a nitrocatecol of formula I, may exhibit a bulk density of less than 0.1 g/ml prior to granulating. Compositions according to the present disclosure comprising 2,5-dichloro-3-(5-(3,4-dihydroxy-5-nitrophenyl)-1,2,4-oxadiazol-3-yl)-4,6-dimethylpyridine 1-oxide may exhibit bulk densities of 0.2 g/ml or greater, for example 0.4 g/ml or greater, or 0.5 g/ml or greater. In at least one embodiment of the disclosure, compositions of the present disclosure for use as final blends for capsule filling or tabletting comprising 2,5-dichloro-3-(5-(3,4-dihydroxy-5-nitrophenyl)-1,2,4-oxadiazol-3-yl)-4,6-dimethylpyridine 1-oxide may also exhibit bulk densities of 0.2 g/ml or greater, for example 0.4 g/ml or greater, 0.5 g/ml or greater, and 0.6/ml or greater.

In certain embodiments of the disclosure, compressed formulations of the disclosure such as tablets exhibit apparent density of 0.5 g/mL to 1.5 g/mL, such as 0.6 g/mL to 1.4 g/mL, 0.7 g/mL to 1.3 g/mL, or 0.8 g/mL to 1.2 g/mL.

The apparent density of a compressed formulation is measured in terms of mass and volume of the formulation and is within the capabilities of the skilled person.

It is also within the ability of one of skill in the art to determine the flowability/flow rate of a compound or composition using methods generally accepted in the art. However, suitable methods include, for example, testing the flow rate through an orifice described in USP 31, vol. 1, test <1174>, The United States Pharmacopeia Convention, 2008. The flowability may be measured as the mass per time flowing through the 10 mm diameter opening of a glass funnel. A flow rate of value greater than 10 g/second is considered good whereas a value of less than 10 g/second is considered poor.

The compressibility index and Hausner ratio are also suitable methods to assess the compound or compositions. For example, the compressibility index and Hausner ratio may be assessed using USP 31, vol. 1, test <1174>, The United States Pharmacopeia Convention, 2008, and measuring both the bulk volume (V₀) and the tapped volume (V_{f}) of the granules. The compressibility index (CI) may then be calculated using the following formula:

CI(%)=100×[V₀−V_{f})/V₀]

The Hausner ratio (HR) can be calculated by using the following formula:

HR=V₀/V_{f}

A compressibility index is considered good when a value of less than 15% is calculated. A Hausner ratio value (a measure of flowability) is considered good when a value of less than 1.25 is calculated.

In various exemplary embodiments of the present disclosure, the compositions and/or formulations may be stable and/or exhibit enhanced stability over other compositions and/or formulations. As used herein, the terms “stability,” “stable,” and variations thereof, is intended to mean that less than 15% of the at least one API in the composition and/or formulation decomposes over 6 months at test conditions of 40° C. and 75% relative humidity, or over 3 years at test conditions of 25° C. or 30° C. and 60% relative humidity or over 15-30 days at test conditions of 70° C. and uncontrolled humidity. In various embodiments, for example, less than 10 wt %, less than 8 wt %, less than 6 wt %, less than 5 wt %, less than 4 wt %, less than 3 wt %, less than 2 wt %, or less than 1 wt % of the at least one API may decompose under these conditions. It is within the ability of one of skill in the art to determine the stability of a compound, composition, or formulation using methods generally accepted in the art. For example, the amount of the at least one API may be measured by any suitable method, e.g., HPLC. For example, in various embodiments, the assay (i.e., the amount of API) of a stable composition or formulation may indicate 85-115% of API after testing conditions, such as 95-105% of API

Decomposition is a chemical process made up of at least one reaction, such as oxidation, reduction or hydrolysis, that results in a chemical change in the decomposing substance resulting in the generation of one or more new chemical compounds. These new compounds (or impurities) may result in reduced and/or variable amount of the API in a given composition and/or formulation, reducing its efficiency, and may have unwanted and/or harmful side effects on the patients. As used herein the term “impurity” means any such new compound that is present in the composition and/or formulation in an amount less than 10 wt % of the API, for example less than 5 wt %, less than 3 wt %, less than 1 wt %, or less than 0.5 wt % of the API. Thus, the change in total impurities in the composition and/or formulation under the conditions and time periods set forth herein may also be indicative of a stable composition or formulation and may be measures by a suitable method, e.g., HPLC. In various embodiments, for example, the total impurities relative to the API in a stable composition and/or formulation after testing conditions may increase by less than 5 wt %, less than 2 wt %, less than 1 wt % or less than 0.5 wt %,

Stability may also be tested under the influence of a variety of other test conditions, including, for example:
40° C. at 75% relative humidity for 6 months;
25° C. or 30° C. at 60% relative humidity after 3-5 years (long-term conditions); and
70° C. at uncontrolled humidity after 15-30 days (stress conditions).

Stability may also be determined by appearance. As used herein, the term “visual stability,” and variations thereof, is intended to mean insubstantial changes in the color, integrity of a compressed formulation (for example, not breaking up), shape, and/or size of the granules, composition and/or formulation.

As used herein, the term “enhanced stability,” “improved stability” and variations thereof, means that the amount of decomposition of the at least one API in a given composition and/or formulation, and/or the increase in impurities in a given composition and/or formulation is less than that of a comparative composition and/or formulation that has been subject to the test conditions.

Unless otherwise indicated, all numbers used in the specification and claims are to be understood as being modified in all instances by the term “about,” whether or not so stated. It should also be understood that the precise numerical values used in the specification and claims form additional embodiments of the disclosure. Efforts have been made to ensure the accuracy of the numerical values disclosed in the Examples. Any measured numerical value, however, can inherently contain certain errors resulting from the standard deviation found in its respective measuring technique.

As used herein the use of “the,” “a,” or “an” means “at least one,” and should not be limited to “only one” unless explicitly indicated to the contrary. Thus, for example, the use of “the formulation” or “a formulation” is intended to mean at least one formulation.

Other embodiments of the disclosure will be apparent to those skilled in the art from consideration of the specification and practice of the present disclosure. It is intended that the specification and examples be considered as exemplary only, with a true scope and spirit of the invention being indicated by the claims.

### EXAMPLES

The following examples are not intended to be limiting of the invention as claimed.

### Example 1

Four low dosage capsules were made on a pilot batch scale by first mixing the API, starches, and lactose in the amounts set forth in Table 1 below (batches A-D). The API used in these examples was 2,5-dichloro-3-(5-(3,4-dihydroxy-5-nitrophenyl)-1,2,4-oxadiazol-3-yl)-4,6-dimethylpyridine 1-oxide. Purified water was then added to each mixture, and the mixtures were granulated by mixing.

The granules were then dried using a fluid bed dryer until a loss on drying value of the granule was below 6%. The dried granules were sieved and then blended with the remaining ingredients set forth in Table 1. Gelatin capsules were filled with the formulation using an InCAP HS capsule filling machine.

The granules and final compositions were evaluated for bulk and tapped density using the methods described above. Flowability/flow rate was also assessed by testing the flow rate through an orifice described in USP 31, vol. 1, test <1174>, The United States Pharmacopeia Convention, 2008. The flowability was measured as the mass per time flowing through the 10 mm diameter opening of a glass funnel. A flow rate of value greater than 10 g/second is considered good whereas a value of less than 10 g/second is considered poor.

The compressibility index and Hausner ratio were assessed using USP 31, vol. 1, test <1174>, The United States Pharmacopeia Convention, 2008, and measuring both the bulk volume (V₀) and the tapped volume (V_{f}) of the granules. The compressibility index (CI) was then calculated using the following formula:

CI(%)=100×[V₀−V_{f})/V₀]

The Hausner ratio (HR) can be calculated by using the following formula:

HR=V₀/V_{f}

A compressibility index is considered good when a value of less than 15% is calculated. A Hausner ratio value (a measure of lowability) is considered good when a value of less than 1.25 is calculated.

Moisture or dryness was determined by loss on drying as described in USP 31, vol. 1, test <731>, The United States Pharmacopeia Convention, 2008. The test involves accurately weighing the substance to be tested (m₀), (e.g. using a sample amount of 1 to 2 g). The test specimen is then dried at 105° C. until a constant weight (m_{f}) is achieved. The moisture can be calculated by using the following expression:

LOD(%)=[(mₒ−m_{f})/m₀]*100

Capsules were evaluated for uniformity of mass and impurities. Uniformity of mass was assessed by the individual weight of 20 capsules; average mass and standard deviation were then calculated. Amount of total impurities was obtained using HPLC method with a limit of quantification of below 0.05%.

The results are set forth in Table 2 below. All batches presented good granule and capsule properties.

### Example 2

Four high dosage capsules were made on a laboratory scale by first mixing the API, starches, and lactose in the amounts set forth in Table 1 below (batches E-H) in a V-blender. The API used in these examples was 2,5-dichloro-3-(5-(3,4-dihydroxy-5-nitrophenyl)-1,2,4-oxadiazol-3-yl)-4,6-dimethylpyridine 1-oxide. Purified water was added to each mixture and mixed manually. The wet mass thus obtained was then granulated in an oscillation granulator laboratory.

The granules were then dried in a tray dryer until a loss on drying of the granule was below 6%. The dried granules were sieved. The granules were then blended with the remaining ingredients set forth in Table 1 in a V-blender. Gelatin capsules were filled with the formulation using an InCAP HS capsule filling machine.

Each of Batch E-H was evaluated as set forth in Example 1 above and the results are set forth in Table 3 below. All batches presented good granule and capsule properties.

TABLE 1
Batch Formulations

| | BATCH | | | | | | | |
|---|---|---|---|---|---|---|---|---|
| Ingredient (%/capsule) | A | B | C | D | E | F | G | H |
| API 9-1067 | 0.4 | 0.4 | 0.4 | 0.4 | 40 | 40 | 40 | 40 |
| Maize starch | 0 | 82.0 | 40.8 | 26.0 | 0 | 42.4 | 21.2 | 14.0 |
| Lactose 200 | 82.0 | 0 | 41.2 | 56.0 | 42.4 | 0 | 21.2 | 28.4 |
| Starch 1500 ™ | 6.8 | 6.8 | 6.8 | 6.8 | 6.8 | 6.8 | 6.8 | 6.8 |
| Explotab ™ | 6.8 | 6.8 | 6.8 | 6.8 | 6.8 | 6.8 | 6.8 | 6.8 |
| Purified water | q.ad | q.ad | q.ad | q.ad | q.ad | q.ad | q.ad | q.ad |
| Silica colloidal hydrated (Syloid ™) | 2.0 | 2.0 | 2.0 | 2.0 | 2.0 | 2.0 | 2.0 | 2.0 |
| Talc | 2.0 | 2.0 | 2.0 | 2.0 | 2.0 | 2.0 | 2.0 | 2.0 |
| capsule size 1 | 1 un | 1 un | 1 un | 1 un | 1 un | 1 un | 1 un | 1 un |

TABLE 2
Analytical Results for Batches A-D

BATCH

| | | | |
|---|---|---|---|
| A | B | C | D |

Granule results

| | | | | |
|---|---|---|---|---|
| Bulk density (g/ml) | 0.62 | 0.59 | 0.60 | 0.64 |
| Tapped density 1250 | 0.75 | 0.73 | 0.70 | 0.74 |
| (g/ml) | | | | |
| Compressibility index | Good | Good | Good | Good |
| Hausner ratio | Good | Good | Good | Good |
| Flow rate | Good | Good | Good | Good |

Capsule results

| | | | | |
|---|---|---|---|---|
| Uniformity mass | 2.39 | 1.77 | 1.26 | 1.14 |
| (RSD %) | | | | |
| Impurities (%) | 3.34 | 3.30 | 3.23 | 3.38 |

TABLE 3
Analytical Results for Batches E-H

BATCH

| | | | |
|---|---|---|---|
| E | F | G | H |

Granule results

| | | | | |
|---|---|---|---|---|
| Bulk density (g/ml) | 0.60 | 0.57 | 0.55 | 0.56 |
| Tapped density 1250 | 0.68 | 0.62 | 0.60 | 0.64 |
| (g/ml) | | | | |
| Compressibility index | Good | Good | Good | Good |
| Hausner Ratio | Good | Good | Good | Good |
| Flow rate | Good | Poor | Poor | Poor |

Capsule results

| | | | | |
|---|---|---|---|---|
| Uniformity mass | 2.86 | 2.34 | 2.08 | 2.89 |
| (RSD %) | | | | |
| Impurities (%) | 2.95 | 3.07 | 2.98 | 2.97 |

### Example 3

All batches from Examples 1 and 2 were put on stress studies to determine their stability. Each of the eight batches was stored for 15 days at room temperature as well as and under stress conditions (70° C. without relative humidity control). All batches were tested for impurities content for both storage conditions, the results of which are set forth in Tables 4 and 5. Impurities values were obtained using HPLC method with a limit of quantification of below 0.05%.

API used in these batches contained around 3% of impurities prior to formulation (composed of impurity 8).

TABLE 4
Results from Stability Testing on Batches A-D

| | A | | B | | C | | D | |
|---|---|---|---|---|---|---|---|---|
| | Dosage | | | | | | | |
| | Low | | Low | | Low | | Low | |
| Storage | RT | SC | RT | SC | RT | SC | RT | SC |
| Total | 3.34 | 3.89 | 3.30 | 5.04 | 3.23 | 5.36 | 3.39 | 4.03 |
| Impurities (%) | | | | | | | | |
| Impurity 8 | 3.34 | 3.03 | 3.30 | 2.90 | 3.23 | 3.29 | 3.39 | 3.17 |
| Impurity 1 | <0.05 | 0.76 | <0.05 | 1.37 | NP | 1.38 | NP | 0.68 |
| Impurity 2 | NP | 0.06 | NP | 0.05 | NP | 0.19 | NP | 0.11 |
| Impurity 3 | NP | NP | NP | 0.07 | NP | 0.14 | NP | NP |
| Impurity 4 | NP | NP | NP | 0.20 | NP | 0.10 | <0.05 | <0.05 |
| Impurity 5 | NP | NP | NP | 0.18 | NP | 0.15 | NP | NP |
| Impurity 6 | NP | 0.07 | NP | 0.14 | NP | 0.14 | NP | 0.07 |
| Impurity 7 | NP | <0.05 | NP | 0.12 | NP | 0.13 | NP | NP |

RT—room temperature
SC—Stress conditions
NP—not present (below detection limit)

TABLE 5
Results from Stability Testing on Batches E-H

| | E | | F | | G | | H | |
|---|---|---|---|---|---|---|---|---|
| | Dosage | | | | | | | |
| | High | | High | | High | | High | |
| Storage | RT | SC | RT | SC | RT | SC | RT | SC |
| Total | 2.96 | 2.86 | 3.07 | 3.03 | 2.98 | 2.78 | 2.97 | 2.78 |
| Impurities (%) | | | | | | | | |
| Impurity 8 | 2.96 | 2.78 | 3.07 | 2.94 | 2.98 | 2.71 | 2.97 | 2.75 |
| Impurity 1 | 0.06 | 0.08 | NP | 0.10 | NP | 0.07 | NP | 0.06 |
| Impurity 2 | NP | <0.05 | NP | <0.05 | NP | NP | NP | NP |
| Impurity 3 | NP | NP | NP | NP | NP | NP | NP | NP |
| Impurity 4 | <0.05 | <0.05 | <0.05 | <0.05 | NP | NP | NP | NP |
| Impurity 5 | NP | NP | NP | NP | NP | NP | NP | NP |
| Impurity 6 | NP | NP | NP | NP | NP | NP | NP | NP |
| Impurity 7 | NP | NP | NP | NP | NP | NP | NP | NP |

RT—room temperature
SC—Stress conditions
NP—not present (below detection limit)

### Example 4

Batch E from Example 2 was put on long term stability studies to determine its stability. In one study, the batch was stored for 6 months at 25° C. and 60% relative humidity, and in a second study, the batch was stored for 6 months at 40° C. and 75% relative humidity. After each test, the batch was tested for assay and impurities content, the results of which are set forth in Table 6. Assay and impurities values were obtained using HPLC method with a limit of quantification of below 0.05%.

TABLE 6
Stability Data for Batch E

| Batch | E | | |
|---|---|---|---|
| Time | 0 | 6 months | 6 months |
| Storage | | 25° C./60% RH | 40° C./75% RH |
| Assay (%) | 96 | 99 | 98 |
| Change in Total | <0.05 | <0.05 | <0.05 |
| Impurities | | | |
| Content (%) | | | |

### Comparative Examples

Three intermediate dosage capsules were made by first mixing the API, the filler(s) the binder and the disintegrant (smaller portion in comparative example and the total amount in batches I and J) in the amounts set forth in Table 7 below for 3 minutes in a high shear mixer. The API used in these examples was 2,5-dichloro-3-(5-(3,4-dihydroxy-5-nitrophenyl)-1,2,4-oxadiazol-3-yl)-4,6-dimethylpyridine 1-oxide. Purified water was added to each mixture over a 3 minute period, and the mixtures were granulated by mixing for an additional 3 minutes.

The granules were then dried in a fluid bed dryer until a loss on drying value of the granule was below 6%. The dried granules were sieved and then blended with the remaining ingredients set forth in Table 7 in a biconic blender. Gelatin capsules were filled with the formulation using an InCAP HS capsule filling machine.

TABLE 7
Formulations for Comparative Example 1

| | BATCH | | |
|---|---|---|---|
| Ingredient (%/capsule) | Comp. | I | J |
| API | 2 | 2 | 2 |
| Di-Calcium-Phosphate | 33 | | |
| (filler) | | | |
| Lactose(filler) | | 80 | 54 |
| Microcrystalline | 46 | | |
| Cellulose(filler) | | | |
| Croscarmellose-Sodium | 2 | | |
| (disintegrant) | | | |
| Maize starch (filler) | | | 27 |
| Povidone (binder) | 7 | | |
| Purified Water | q.s. | q. ad | q. ad |
| Modified starch (Binder) | | 7 | 7 |
| Sodium amidoglycolate | | 7 | 7 |
| (Disintegrant) | | | |
| Croscarmellose-Sodium | 4 | | |
| (disintegrant) | | | |
| Silica Colloidal Hydrate | 4 | 2 | 2 |
| (lubricant) | | | |
| Talc (lubricant) | 2 | 2 | 2 |
| Magnesium-Stearate | 2 | | |
| (lubricant) | | | |

The granules and capsules were evaluated and results are shown in Table 8 below. After two stability studies, one under 25° C. and 60% RH and the other under 40° C. and 75% RH for 6 months each, it was observed that batches I and J exhibit enhanced stability when compared with the comparative composition.

TABLE 8
Stability of Formulae after 6 Months at 40° C. at 75% RH

| | Batch | | | | | | | | |
|---|---|---|---|---|---|---|---|---|---|
| | Comparative (Comp.) | | | I | | | J | | |
| | Time months) | | | | | | | | |
| | 0 | 6 | 6 | 0 | 6 | 6 | 0 | 6 | 6 |
| Storage | | 25° C./ | 40° C./ | | 25° C./ | 40° C./ | | 25° C./ | 40° C./ |
| | | 60% RH | 75% RH | | 60% RH | 75% RH | | 60% RH | 75% RH |
| Assay (%) | 99 | 99 | 92 | 98 | 100 | 102 | 97 | 98 | 100 |
| Change in | | 0.07 | 2.34 | | None | 0.15 | | None | 0.15 |
| Total | | | | | detected | | | detected | |
| Impurities | | | | | | | | | |
| Content | | | | | | | | | |

1. A stable composition comprising:
at least one active pharmaceutical ingredient (API) chosen from 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol and salts, esters, hydrates, solvates and other derivatives thereof;
at least one filler; and
at least one binder;
wherein at least the at least one active pharmaceutical ingredient is present in the composition in granular form.

2. The stable composition of claim 1, wherein the at least one API is a combination of 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol.

3. The stable composition of claim 1, wherein less than 10% of the API decomposes over 15 days of storage at 70° C. and uncontrolled humidity, over 6 months at 40° C. and 75% relative humidity, and/or over 3 years at 60% relative humidity and 30° C. or 25° C.

4-6. (canceled)

7. The stable composition of claim 1, wherein the increase in total impurities is less than 5% over 15 days of storage at 70° C. and uncontrolled humidity, over 6 months at 40° C. and 75% relative humidity, and/or over 3 years at 60% relative humidity and 30° C. or 25° C.

8-10. (canceled)

11. The stable composition of claim 1, wherein the at least one filler is chosen from lactose, maize starch and microcrystalline cellulose.

12. The stable composition of claim 1, wherein the at least one binder is chosen from hypromellose, hydroxypropyl cellulose, methyl- or ethyl-cellulose, pregelatinized maize starch and gelatin.

13. The stable composition of claim 1, wherein the granules further comprise the at least one filler and/or at least one binder.

14. The stable composition of claim 1, further comprising at least one additional excipient chosen from disintegrants, glidants, and lubricants.

15. The stable composition of claim 1, wherein the composition exhibits improved bulk density.

16. The stable composition of claim 15 wherein the composition exhibits a bulk density of greater than 0.1 g/ml.

17-22. (canceled)

23. A pharmaceutical formulation comprising a stable composition comprising:
at least one active pharmaceutical ingredient (API) chosen from 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol and salts, esters, hydrates, solvates and other derivatives thereof;
at least one filler; and
at least one binder;
wherein at least the at least one active pharmaceutical ingredient is present in the composition in granular form.

24. The pharmaceutical formulation of claim 23, wherein the formulation is in the dosage form of a tablet or capsule.

25. The pharmaceutical formulation of claim 23, wherein the pharmaceutical formulation is stable.

26. The pharmaceutical formulation of claim 25, wherein less than 10% of the API decomposes over 15 day of storage at 70° C. and uncontrolled humidity, over 6 months at 40° C. and 75% relative humidity, and/or over 3 years at 60% relative humidity and 30° C. or 25° C.

27-29. (canceled)

30. A method of manufacturing a stable pharmaceutical formulation, said method comprising:
granulating at least one active pharmaceutical ingredient (API) chosen from 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol and salts thereof to form granules;
mixing at least one filler with the at least one active pharmaceutical ingredient before, during or after granulation;
mixing at least one binder with the at least one active pharmaceutical ingredient before, during or after granulation; and
preparing a pharmaceutical formulation in the form of a dosage form.

31. The method of claim 30, wherein the at least one API is a combination of 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol.

32. The method of claim 30, wherein the at least one filler is chosen from lactose, maize starch and microcrystalline cellulose.

33. The method of claim 30 wherein the at least one binder is chosen from hypromellose, hydroxypropyl cellulose, methyl- or ethyl-cellulose, pregelatinized maize starch and gelatin.

34. The method of claim 30, wherein less than 10% of the API in the dosage form decomposes over 15 days of storage at 70° C. and uncontrolled humidity, over 6 months at 40° C. and 75% relative humidity, and/or over 3 years at 60% relative humidity and 30° C. or 25° C.

35-37. (canceled)

38. The method of claim 30 wherein the increase in total impurities is less than 5% over 15 days of storage at 70° C. and uncontrolled humidity, over 6 months at 40° C. and 75% relative humidity, and/or over 3 years at 60% relative humidity and 30° C. or 25° C.

39-42. (canceled)

43. The method of claim 30, wherein the granulation process is wet granulation.

44-47. (canceled)

48. The method of claim 30, wherein the granules and/or composition exhibit improved bulk density.

49. The method of claim 48, wherein the granules and/or composition exhibit a bulk density greater than 0.1 g/ml.

50-120. (canceled)

121. The stable composition of claim 1, wherein the at least one filler is not a phosphate-derivative; and
wherein the at least one binder is not a PVP derivative compound.

122. The stable composition of claim 1, wherein the active pharmaceutical ingredient is present in a therapeutically effective amount.

123. The stable composition of claim 1, wherein the composition further comprises an additional active pharmaceutical ingredient.

124. The stable composition of claim 121, wherein the composition further comprises at least one additional excipient chosen from disintegrants, glidants, and lubricants.

125. The pharmaceutical formulation of claim 23, wherein the at least one filler is not a phosphate-derivative; and
wherein the at least one binder is not a PVP derivative compound.

126. The pharmaceutical formulation of claim 23, wherein the granules further comprise the at least one filler and/or at least one binder.

127. The pharmaceutical formulation of claim 23, wherein the composition exhibits improved apparent density.

128. The pharmaceutical formulation of claim 127, wherein the composition exhibits an apparent density greater than 0.1 g/ml.

129. The pharmaceutical formulation of claim 23, wherein the at least one active pharmaceutical ingredient is a combination of 2,5-dichloro-3-(5-(3,4-dihydroxy-5-nitrophenyl)-1,2,4-oxadiazol-3-yl)-4,6-dimethylpyridine 1-oxide and 5-[3-(2,5-dichloro-4,6-dimethylpyridin-3-yl)-[1,2,4]oxadiazol-5-yl]-3-nitrobenzene-1,2-diol.

130. The pharmaceutical formulation of claim 25, wherein the increase in total impurities is less than 5% over 15 days of storage at 70° C. and uncontrolled humidity, over 6 months at 40° C. and 75% relative humidity, and/or over 3 years at 60% relative humidity and 30° C. or 25° C.
